SEQUENCE LISTING (19 sequences)

SEQ ID NO: 1-7 - skipped (7 entries)

SEQ ID NO: 8 - DNA, 111 bp, synthetic construct
  source 1..111: mol_type = other DNA
gaaattaatacgactcactatagggttgttggggtaaccaactatttgttactgttgtttatgtcattatgtgctgccatatctacttcagaaactacatataaaaatact

SEQ ID NO: 9 - DNA, 111 bp, synthetic construct
  source 1..111: mol_type = other DNA
agtatttttatatgtagtttctgaagtagatatggcagcacataatgacataaacaacagtaacaaatagttggttaccccaacaaccctatagtgagtcgtattaatttc

SEQ ID NO: 10 - protein, 1152 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 11 - protein, 1120 aa, synthetic construct
MKNIQRLGKGNEFSPFKKEDKFYFGGFLNLANNNIEDFFKEIITRFGIVITDENKKPKETFGEKILNEIFKKDISIVDYEKWVNIFADYFPFTKYLSLYLEEMQFKNRVICFRDVMKELLKTVEALRNFYTHYDHEPIKIEDRVFYFLDKVLLDVSLTVKNKYLKTDKTKEFLNQHIGEELKELCKQRKDYLVGKGKRIDKESEIINGIYNNAFKDFICKREKQDDKENHNSVEKILCNKEPQNKKQKSSATVWELCSKSSSKYTEKSFPNRENDKHCLEVPISQKGIVFLLSFFLNKGEIYALTSNIKGFKAKITKEEPVTYDKNSIRYMATHRMFSFLAYKGLKRKIRTSEINYNEDGQASSTYEKETLMLQMLDELNKVPDVVYQNLSEDVQKTFIEDWNEYLKENNGDVGTMEEEQVIHPVIRKRYEDKFNYFAIRFLDEFAQFPTLRFQVHLGNYLCDKRTKQICDTTTEREVKKKITVFGRLSELENKKAIFLNEREEIKGWEVFPNPSYDFPKENISVNYKDFPIVGSILDREKQPVSNKIGIRVKIADELQREIDKAIKEKKLRNPKNRKANQDEKQKERLVNEIVSTNSNEQGEPVVFIGQPTAYLSMNDIHSVLYEFLINKISGEALETKIVEKIETQIKQIIGKDATTKILKPYTNANSNSINREKLLRDLEQEQQILKTLLEEQQQREKDKKDKKSKRKHELYPSEKGKVAVWLANDIKRFMPKAFKEQWRGYHHSLLQKYLAYYEQSKEELKNLLPKEVFKHFPFKLKGYFQQQYLNQFYTDYLKRRLSYVNELLLNIQNFKNDKDALKATEKECFKFFRKQNYIINPINIQIQSILVYPIFLKRGFLDEKPTMIDREKFKENKDTELADWFMHYKNYKEDNYQKFYAYPLEKVEEKEKFKRNKQINKQKKNDVYTLMMVEYIIQKIFGDKFVEENPLVLKGIFQSKAERQQNNTHAATTQERNLNGILNQPKDIKIQGKITVKGVKLKDIGNFRKYEIDQRVNTFLDYEPRKEWMAYLPNDWKEKEKQGQLPPNNVIDRQISKYETVRSKILLKDVQELEKIISDEIKEEHRHDLKQGKYYNFKYYILNGLLRQLKNENVENYKVF

SEQ ID NO: 12 - RNA, 64 nt, synthetic construct
  source 1..64: mol_type = other RNA
gatttagactaccccaaaaacgaaggggactaaaactctgaagtagatatggcagcacataatg

SEQ ID NO: 13 - DNA, 66 bp, synthetic construct
  source 1..66: mol_type = other DNA
tctgaagtagatatggcagcacataatgacgttggaactgctctcattttggagggtaatcacaac

SEQ ID NO: 14 - DNA, 20 bp, synthetic construct
  source 1..20: mol_type = other DNA
taatacgactcactataggg

SEQ ID NO: 15 - DNA, 20 bp, synthetic construct
  source 1..20: mol_type = other DNA
ccctatagtgagtcgtatta

SEQ ID NO: 16 - DNA, 372 bp, synthetic construct
  source 1..372: mol_type = other DNA
atggctgatccagaaggtacagacggggagggcacgggttgtaacggctggttttatgtacaagctattgtagacaaaaaaacaggagatgtaatatctgatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcaaagaaaacagtccattaggggagcggctggaggtggatacagagttaagtccacggttacaagaaatatctttaaatagtgggcagaaaaag

SEQ ID NO: 17 - DNA, 54 bp, synthetic construct
  source 1..54: mol_type = other DNA
gaaattaatacgactcactatagggtcggatatggtagattttattgatacaca

SEQ ID NO: 18 - DNA, 30 bp, synthetic construct
  source 1..30: mol_type = other DNA
catgcaacacttgtgcatcattgtggacct

SEQ ID NO: 19 - RNA, 66 nt, synthetic construct
  source 1..66: mol_type = other RNA
tgctgtctctagctctgcctgttcacaaaagttggaactgctctcattttggagggtaatcacaac

(19) United States
(12) Patent Application Publication
(10) Pub. No.: US 2025/0305039 A1
(43) Pub. Date: Oct. 2, 2025

(54) METHOD FOR DETECTING TARGET NUCLEIC ACID BY CLEAVING NON-NATURAL SEQUENCE USING CAS13 PROTEIN

(71) Applicant: Orange Biotech Limited, Pak Shek Kok (HK)

(72) Inventors: Xin LIU, Pak Shek Kok (HK); Shuhuai YAO, Pak Shek Kok (HK); Christopher CS CHAN, Pak Shek Kok (HK)

(21) Appl. No.: 19/085,731

(22) Filed: Mar. 20, 2025

Related U.S. Application Data
(60) Provisional application No. 63/570,953, filed on Mar. 28, 2024.

(51) Int. Cl. C12Q 1/6853; C12N 9/22; C12N 15/113; C12Q 1/6876

(52) U.S. Cl. CPC C12Q 1/6853; C12N 9/226; C12N 15/113; C12Q 1/6876; C12N 2310/3519

(57) ABSTRACT

The present invention provides a method for detecting a target nucleic acid by cleaving a non-natural sequence using a Cas13 protein, belonging to the technical field of biology. The Cas13 protein belongs to the Cas13b protein family; and the non-natural sequence includes a chimeric sequence composed of a ribonucleotide and a deoxyribonucleotide. The present invention provides a system for detecting a target nucleic acid. The system includes a chimeric sequence and a Cas13 protein guided by crRNA. Moreover, it is verified that the detection effect of the Cas13-chimeric sequence system is comparable to that of a conventional Cas13-ssRNA system, and the former is even superior to the latter under certain circumstances. The present invention combines amplification technology with the system, enabling the detection limit of the system to reach an aM level. In summary, the present invention provides a new option for the field of nucleic acid detection, and meanwhile also broadens the use of the Cas13 protein and the non-natural sequence.

### CROSS-REFERENCE TO RELATED APPLICATIONS

The present application claims priority to U.S. prior provisional application No. 63/570,953 filed on Mar. 28, 2024, the claims, specification, drawings of specification, and abstract of which are incorporated herein by reference in their entirety as part of the present invention.

### REFERENCE TO AN ELECTRONIC SEQUENCE LISTING

The content of the electronic sequence listing (Sequence Listing.xml; Size: 16,119 bytes; and Date of Creation: Apr. 16, 2025) is herein incorporated by reference in its entirety.

### BACKGROUND OF THE INVENTION

### Field of the Invention

The present invention relates to the field of biotechnology, and in particular to a method for detecting a target nucleic acid by cleaving a non-natural sequence using a Cas13 protein.

### Description of the Related Art

Due to the programmability and sensitivity of the clustered regularly interspaced short palindromic repeats (CRISPR) and the matched Cas proteins, the CRISPR-Cas system has become a promising technology in the field of isothermal nucleic acid detection (1). Among different types of Cas proteins, Class II proteins such as Cas13 and Cas12 not only have the function of specifically recognizing target molecules (2-3), but also exhibit other activities. That is, once the Cas proteins bind to the target sequence under the guidance of CRISPR RNA (crRNA), these Cas proteins will degrade their surrounding nucleotide sequences. Traditionally, the Cas12 system is considered to be a system that targets DNA and trans-cleaves DNA, while the Cas13 system is a system that targets RNA and trans-cleaves RNA (4-5).

The latest research shows that Cas12 proteins can not only cleave DNA sequences, but also cleave RNA (6-7). In addition, DNA helper sequences can be introduced to assist Cas12a in cleaving RNA sequences. This indicates that the Cas12a protein can perform its cleavage function without having to be completely matched with the target DNA sequence (8). In contrast, the Cas13 protein is believed to be capable of only recognizing and trans-cleaving RNA molecules. It is worth noting that the Cas13 protein shows a preference for RNA motifs composed of two types of ribonucleotides, suggesting that the Cas13 protein has different mechanisms of action when trans-cleaving different types of nucleic acids (9).

Nucleic acid sequences include natural nucleic acid sequences (such as DNA and RNA) and non-natural nucleic acid sequences (such as chimeric sequences and XNA (xeno nucleic acids)). The non-natural sequences are composed of elements that exist in nature or have been modified. The elements include nucleobases, glycosyl groups, and phosphate backbones that are different from those in the structures of DNA and RNA. Synthetic nucleic acid sequences have higher biochemical stability (10-11) and are capable of storing genetic information (12). In addition, enzymes capable of degrading XNA are currently underdevelopment (13).

Current research has revealed that the Cas12 protein family can achieve the purpose of detection and reporting by cleaving non-natural sequences. However, it is still unclear whether the Cas13 nuclease can also trans-cleave unconventional RNA sequences.

### BRIEF SUMMARY OF THE INVENTION

Traditional nucleic acid detection systems based on the Cas13a protein rely on the ability of the Cas13a protein to trans-cleave RNA, and modifications are made to the cleaved substrate to detect the process of Cas13a cleaving RNA. Such modifications include, but are not limited to, fluorescent groups, biotin, or other physical labels. Although it is currently known that the Cas13a protein can cleave single-stranded RNA (ssRNA), it is still unknown whether it can cleave other forms of nucleic acid sequences. Moreover, there are few studies on the trans-cleavage activities of other members of the Cas13 protein family.

The present invention explores the above issues and finds that the LwCas13a protein only shows RNase activity, that is, it can only degrade conventional RNA but cannot degrade chimeric sequences. In contrast, the CcaCas13b protein exhibits non-canonical trans-cleavage activity, that is, it can cleave chimeric sequences composed of deoxynucleotides and ribonucleotides, and these chimeric sequences belong to non-natural sequences. Therefore, the present invention reveals that the Cas13b protein has relatively broad trans-cleavage activity on nucleic acids, that is, it has a preference for cleaving non-natural sequences, rather than being limited to RNase activity. This characteristic enables the Cas13b protein to target and cleave non-natural sequences, thereby facilitating the development of new use. Next, the present invention further confirms that the ability of the Cas13b protein to trans-cleave non-natural sequences reaches the level of its cleavage of traditional RNA. Furthermore, the present invention utilizes the activity of the Cas13b protein to cleave non-natural sequences to construct a novel detection system (i.e., the Cas13b-chimeric sequence system), and combines this detection system with the recombinase polymerase amplification technology (RPA) to further improve the detection sensitivity of the system, enabling its sensitivity to reach the aM level. In addition, the system can complete the detection within 1 hour. In summary, on the one hand, the present invention broadens the understanding of those skilled in the art regarding the subtypes of the Cas13 protein. That is, the Cas13 protein not only possesses RNase activity but also has the ability to trans-cleave non-natural sequences. Based on this characteristic, a novel CRISPR detection system is designed, and the system is efficient and sensitive. On the other hand, the present invention also broadens the application of non-natural sequences.

In order to achieve the above objectives, the technical solutions adopted in the present invention are as follows.

Aiming at the aforementioned traditional problems, the present invention provides a method or kit and system for detecting presence or quantity of a target nucleic acid by cleaving a non-natural sequence using a Cas13 protein, belonging to the technical field of biology.

It should be understood that in the present invention, the characteristic that the Cas13 protein can trans-cleave the non-natural sequence is referred to as atypical trans-cleavage activity, in order to distinguish it from the typical trans-cleavage activity of nucleases. The typical trans-cleavage activity of nucleases means that nucleases are capable of trans-cleaving natural RNA and/or DNA. The trans-cleavage refers to non-specific cleavage.

It should be noted that the target nucleic acid refers to the nucleic acid (including RNA and DNA) that needs to be detected. However, the Cas13 protein can only be activated by RNA. Therefore, when the target nucleic acid is RNA rather than DNA, it can be directly recognized, bound and detected by the Cas13 protein, while when the target nucleic acid is DNA, the DNA needs to be transcribed into RNA before proceeding with subsequent detection reactions.

In an aspect, the present invention provides use of a Cas13 protein that recognizes RNA under the guidance of crRNA in preparation of a reagent for trans-cleaving a non-natural sequence to detect a target nucleic acid.

The RNA is the target nucleic acid or is transcribed from the target nucleic acid. As described above, since the Cas13 can only be activated by RNA, when the target nucleic acid is RNA, the Cas13 directly recognizes and binds to it. When the target nucleic acid is DNA, the target nucleic acid needs to be transcribed into RNA.

Further, the Cas13 protein includes a Cas13a protein or a Cas13b protein.

Further, the Cas13 protein is a CcaCas13b protein belonging to a Cas13b subfamily.

The non-natural sequence includes a nucleic acid sequence incapable of being produced or incapable of being stably inherited in a long evolutionary process in nature.

Further, the non-natural sequence includes any one or more of the following:
(1) a sequence containing both a deoxynucleotide and a ribonucleotide;
(2) a sequence containing a natural or non-natural deoxynucleotide and/or ribonucleotide, where the deoxynucleotide and/or ribonucleotide bears an artificially created modification that does not exist under natural conditions; and
(3) a sequence containing a deoxynucleotide and/or ribonucleotide, where a backbone composed of the deoxynucleotide and/or ribonucleotide bears an artificially created modification that does not exist under natural conditions.

Further, the non-natural sequence contains both a deoxynucleotide and a ribonucleotide.

Specifically, the non-natural sequence is composed of nucleotides constituting RNA and nucleotides constituting DNA, the nucleotides constituting the RNA are any one or more of a uracil ribonucleotide, an adenine ribonucleotide, a cytosine ribonucleotide and a guanine ribonucleotide, and the nucleotides constituting the DNA are any one or more of a thymine deoxynucleotide, an adenine deoxynucleotide, a cytosine deoxynucleotide and a guanine deoxynucleotide.

Further, the deoxynucleotides and ribonucleotides in the non-natural sequence are arranged alternately at intervals. That is, a deoxynucleotide is followed by a ribonucleotide, or a ribonucleotide is followed by a deoxynucleotide.

Specifically, the non-natural sequence is arranged in the order of ribonucleotide-deoxynucleotide or deoxynucleotide-ribonucleotide, the ribonucleotide is any one or more of a uracil ribonucleotide, an adenine ribonucleotide, a cytosine ribonucleotide and a guanine ribonucleotide, and the deoxynucleotide is any one or more of a thymine deoxynucleotide, an adenine deoxynucleotide, a cytosine deoxynucleotide and a guanine deoxynucleotide.

More specifically, the non-natural sequence includes the following non-natural sequences: any one or more of rUArUArUA, ArUArUArU, rUrUrArUrUrU, TrUTrUTrU, ArAArAArA, CrCCrCCrC and GrGGrGGrG, where the rU is a uracil ribonucleotide, the A is an adenine deoxynucleotide, the T is a thymine deoxynucleotide, the rA is an adenine ribonucleotide, the C is a cytosine deoxynucleotide, the rC is a cytosine ribonucleotide, the G is a guanine deoxynucleotide, and the rG is a guanine ribonucleotide.

More specifically, the non-natural sequence includes any one or more of rUArUArUA and ArUArUArU.

The target nucleic acid is present in a sample. Further, the sample is saliva, blood, urine, or nasal secretions.

The reagent includes the Cas13 protein and the non-natural sequence.

In a second aspect of the present invention, a kit for detecting a target nucleic acid is provided. The kit includes: a Cas13 protein that binds to RNA under the guidance of crRNA, and a non-natural or non-naturally occurring nucleic acid sequence. The non-naturally occurring sequence includes one or more of the following sequences:
(1) a sequence containing both a deoxynucleotide and a ribonucleotide;
(2) a sequence containing a natural or non-natural deoxynucleotide and/or ribonucleotide, where the deoxynucleotide and/or ribonucleotide bears an artificially created modification that does not exist under natural conditions; and
(3) a sequence containing a deoxynucleotide and/or ribonucleotide, where a backbone composed of the deoxynucleotide and/or ribonucleotide bears an artificially created modification that does not exist under natural conditions.

Since only RNA can activate the Cas13 protein, if the detection target is DNA, the DNA needs to be transcribed into RNA first, and then the Cas13 protein can be activated. In some embodiments, the kit also includes a necessary reagent required for a transcription reaction, including an enzyme, crRNA, and an inorganic salt reagent. In some specific examples, using DNA as a template, T7 polymerase is used to synthesize RNA that can activate the Cas13 protein.

In some embodiments, the kit further includes a necessary reagent for amplifying the target nucleic acid, including an enzyme, an inorganic salt, etc. necessary for amplification. In some embodiments, a target nucleic acid amplification manner includes variable-temperature PCR or isothermal amplification, and the isothermal amplification includes LAMP, RPA, RAA and the like. All reagents or components that can amplify the target nucleic acid can be used as an embodiment of the present invention, such as a primer, a probe sequence that binds to the target nucleic acid, etc. In other embodiments, when the object to be detected is RNA and pre-amplification is required, the kit further includes a reagent for reverse-transcribing RNA into cDNA. However, since the Cas13 protein can only recognize RNA, an amplification product also needs to undergo a transcription reaction to generate an RNA activator.

The target nucleic acid here is the nucleic acid to be detected or diagnosed. The nucleic acid is generally a natural nucleic acid sequence or a partial sequence produced by methods such as synthesis and nucleic acid amplification, such as from human tissues, microorganisms (e.g., viruses, bacteria, fungi), and also human or mammalian cells, etc.

In some embodiments, the target nucleic acid is DNA or RNA.

In some embodiments, the DNA or RNA in the target nucleic acid includes double-stranded or single-stranded forms.

In some embodiments, in the target nucleic acid, the DNA is double-stranded and the RNA is single-stranded.

In a third aspect of the present invention, a method for detecting presence or quantity of a target nucleic acid is provided. The method includes: binding a Cas13 protein to a target RNA, and then trans-cleaving a non-natural sequence by the Cas13 protein, so as to detect or identify the presence or quantity of the target nucleic acid from the quantity of the cleaved non-natural sequences.

The target RNA is the target nucleic acid or is transcribed from the target nucleic acid. As described above, since the Cas13 can only be activated by RNA, when the target nucleic acid is RNA, the Cas13 directly recognizes and binds to it. When the target nucleic acid is DNA, the target nucleic acid needs to be transcribed into RNA.

In some embodiments, the non-naturally occurring sequence includes one or more of the following sequences:
(1) a sequence containing both a deoxynucleotide and a ribonucleotide;
(2) a sequence containing a natural or non-natural deoxynucleotide and/or ribonucleotide, where the deoxynucleotide and/or ribonucleotide bears an artificially created modification that does not exist under natural conditions; and
(3) a sequence containing a deoxynucleotide and/or ribonucleotide, where a backbone composed of the deoxynucleotide and/or ribonucleotide bears an artificially created modification that does not exist under natural conditions.

In some embodiments, the non-natural sequence includes a label, and the presence or quantity of the target nucleic acid is detected by detecting the amount or quantity of the label. The label includes fluorescence or any other labeling substances.

In some embodiments, the Cas13 protein belongs to a Cas13a/Cas13b protein family.

In some embodiments, the non-naturally occurring nucleic acid sequence includes a chimeric sequence composed of a ribonucleotide and a deoxyribonucleotide. Such a sequence can be made into a probe for nucleic acid detection. The detection effect of the chimeric sequence is comparable to that of a conventional ssRNA probe, and in specific cases is even better than that of ssRNA. The non-naturally occurring nucleic acid also includes a xeno nucleic acid (XNA), a chimeric sequence, and a hybridized sequence.

In some embodiments, when the target sequence is natural DNA, the DNA needs to be transcribed into RNA.

In some embodiments, the natural target nucleic acid may or may not be amplified. In some embodiments, the target nucleic acid is pre-amplified, and then tested by the method or system of the present invention. The present invention also demonstrates that the pre-amplification of the target nucleic acid can increase the limit of detection of the system to a single-molecule level. In summary, the coordinated use of the Cas13 protein and chimeric sequence pioneers a novel CRISPR/Cas13 detection system and expands the use of the Cas12 protein and non-natural sequence.

In some embodiments, the non-natural or non-naturally occurring nucleic acid of the present invention includes a non-natural or non-naturally occurring nucleic acid sequence in a broad sense, and also includes a non-natural or non-naturally occurring nucleic acid sequence in a narrow sense. In some embodiments, the non-natural or non-naturally occurring nucleic acid of the present invention is the non-natural nucleic acid sequence in a broad sense. In some embodiments, the meanings of the “non-natural sequence” or the “non-naturally occurring sequence” are interchangeable, and they refer to nucleic acid sequences incapable of being produced or incapable of being stably inherited during a long evolutionary process in nature, i.e., other sequences other than natural or naturally occurring DNA and RNA. The natural or naturally occurring nucleic acid sequence (e.g. RNA or DNA) refers to a nucleic acid sequence that can be produced or stably inherited during a long evolutionary process in nature.

In some embodiments, the non-natural sequence refers to a sequence that is different from a natural or naturally occurring DNA/RNA and is created by artificially modifying the components or internal structure of the natural DNA/RNA on the basis of an existing natural DNA/RNA sequence, also known as a xeno nucleic acid (XNA). In some embodiments, the artificial modification of the components of the DNA/RNA includes, but is not limited to, changing the combination manner of the components (i.e., the deoxynucleotides and the nucleotides can appear simultaneously in the same sequence), and making non-natural and artificially created modifications to the internal components of the nucleotides such as pentoses, bases or phosphate groups, etc. The artificial modification methods include, but are not limited to: changing the type of a glycosyl group, introducing an organic polymer and/or a halogen, and a combination of several or multiple of a plurality of methyl or/acetyl modifications. In some embodiments, the artificial base modification can regulate the strength and specificity of base pairing, and the modification of the glycosyl group also has a significant effect on the properties of nucleic acids, e.g. double-strand formation ability, nuclease resistance, and toxicity to cells and animals. In some embodiments, the artificial modification of the internal structure of the DNA/RNA includes, but is not limited to change in a nucleic acid backbone structure (e.g. a thiophosphate), introduction of a new artificial nucleoside (e.g. deoxyuridine), modification of pentoses (e.g. the pentoses of glycol nucleic acids), and deoxy and non-deoxy modifications, etc. In some embodiments, the artificial modification of the phosphodiester backbone can improve the nuclease resistance and pharmacokinetic properties. In a narrow sense, the non-natural sequence refers to a sequence composed of a deoxynucleotide and a ribonucleotide, or a nucleic acid sequence containing a modified deoxynucleotide and/or ribonucleotide and/or sugar-phosphate backbone that is artificially created under non-natural conditions. The meaning of the “non-natural sequence” or “non-naturally occurring sequence” in the present invention includes both the “non-natural sequence” or “non-naturally occurring sequence” in a broad sense and the “non-natural sequence” or “non-naturally occurring sequence” in a narrow sense.

In some embodiments, the xeno nucleic acid (XNA) can store gene information, replicate, and even evolve, just like natural DNA and RNA, but is artificially created or produced, rather than produced by natural evolution. The term “synthetic” in the present invention means artificial synthesis, rather than production in an evolutionary process in nature. The so-called “artificial synthesis” includes the synthesis in which humans participate and take charge, for example synthesis of the “non-natural sequence” or “non-naturally occurring sequence” as defined in the present invention by humans through a machine or in an artificial intelligence (AI) manner.

In some embodiments, the non-natural nucleic acid sequence includes a (rUA)ₙ nucleic acid sequence, where n is any natural integer, and for example, n can be any number from 10,000 to 100 million, e.g., a natural length of 1, 2, 3, 4, 5, 6, 7, 8, 9, 10, 11, 12, 13, 14, 15, etc. In some embodiments, non-natural sequences (e.g., chimeric sequences) in the following forms, such as rUArUArUA, where “rU” represents a uracil ribonucleotide (which only occurs in RNA in vivo) and “A” represents an adenine deoxynucleotide (which only occurs in DNA in vivo), demonstrate how synthetic biology combines natural components to create a novel genetic system. In an embodiment, the non-natural sequence includes an RNA or DNA sequence with (rUA)ₙ units. For example, the natural sequence includes (rUA)ₙ, and for example, a DNA sequence includes a (rUA)ₙ unit, where n is any natural integer. In some embodiments, the natural DNA or RNA sequence includes a nucleic acid sequence with a rUA)ₙ unit, thereby becoming a non-natural sequence or a chimeric sequence.

A basic unit of traditional natural DNA is a deoxyribonucleotide. The deoxyribonucleotide is composed of a base, a deoxyribose and a phosphate. The basic building block of DNA is a deoxyribonucleotide, which is of four types, depending on the base it contains: A (adenine), T (thymine), G (guanine), and C (cytosine). However, the basic building block of natural RNA is a ribonucleotide, which is composed of one molecule of phosphoric acid, one molecule of ribose, and one molecule of nitrogenous base. There are four types of nitrogenous bases: adenine (A), uracil (U), guanine (G) and cytosine (C), respectively.

These chimeric sequences are intermediate states between natural nucleic acids and engineered alternatives, providing new possibilities for the research and application of the present invention. In synthetic biology, non-natural nucleic acids can be used in editable cells, providing new ideas for more stable gene therapies, biosensors, and synthetic organisms. They also have certain potential in the field of biotechnology, where they can be used as probes with a longer shelf life and better durability in molecular diagnostics or as new components of gene editing systems such as CRISPR. Moreover, XNAs can also be used for constructing nanoscale scaffolds or as components of programmable biomolecular systems. More importantly, non-natural nucleic acids can achieve the goal of constructing synthetic cells with completely new genetic information, fundamentally expanding human understanding of lives and potential forms thereof.

In some embodiments, the Cas13 system belongs to a Type VI family, including multiple subtypes such as Cas13a, Cas13b, Cas13c, and Cas13d. The Cas13 protein is a single protein composed of multiple domains, with functions of recognizing crRNA, cleaving RNA, and even cleaving pre-crRNA. The Cas13 protein has 2 signature HEPN domains, where 2 R-XXXX-H conserved motifs are nuclease active sites of the HEPN domains.

The Cas13 protein has both cis-cleavage and trans-cleavage activities. When the Cas13 specifically identifies and cleaves the target nucleic acid (this process is cis-cleavage), such a process can activate its trans-cleavage activity and can cleave any other single-stranded natural RNA in the system into fragments (this process is trans-cleavage) in a short period of time. In the prior art, the trans-cleavage property of the Cas13 is utilized to directly or indirectly detect various target nucleic acids. However, in this solution, it is found that this system can be utilized to trans-cleave non-natural or non-naturally occurring sequences.

The prior art believes that the Cas13 protein can only cleave single-stranded RNAs (either cis- or trans-), and it is still unclear whether it can cleave non-natural sequences. Through extensive research, it is proved in the present invention that members of the Cas13b sub-family among the Cas13 protein can efficiently trans-cleave non-natural sequences, enabling accurate detection of target nucleic acids, thus greatly broadening the application of Cas13b in the field of nucleic acid detection. Specifically, the Cas13b protein is a CcaCas13b protein.

The “trans-cleavage” in the present invention refers to the property that after being activated by the target RNA, the Cas13 protein guided by crRNA can non-specifically cleave any other adjacent nucleic acid sequences. The nucleic acid sequences here include RNA or non-natural sequences.

In some specific examples, the non-natural sequence contains both a deoxynucleotide and a ribonucleotide.

It can be understood that the natural DNA or RNA only contains the deoxynucleotide or the ribonucleotide, so that the non-natural sequence is neither a DNA sequence nor an RNA sequence.

In some specific examples, the deoxynucleotides and ribonucleotides in the non-natural sequence are arranged alternately at intervals. That is, a deoxynucleotide is followed by a ribonucleotide, or a ribonucleotide is followed by a deoxynucleotide. It can be understood that the non-natural sequence does not contain two deoxynucleotides or two ribonucleotides connected by a natural backbone, e.g.: DNA: TT, RNA: UU and the like situations.

The present invention confirms that the Cas13b protein has strong trans-cleavage activity against the non-natural sequence, but there is no report on the research or progress of applying this cleavage ability to nucleic acid detection. The present invention constructs a Cas13b-chimeric sequence detection system and explores its sensitivity. The sensitivity of the Cas13b-chimeric sequence system is comparable to that of the traditional system, reaching the pM level.

The present invention designs 2 non-natural chimeric sequences (rUArUArUA (poly rUA) and ArUArUArU (poly ArU)) and uses the conventional CcaCas13b protein to perform trans-cleavage on them. It is found that the above 2 non-natural sequences can be trans-cleaved by CcaCas13b, where the detection effect of the CcaCas13b-rUArUArUA system is comparable to that of the positive control ssRNA and better than that of the CcaCas13b-ArUArUArU system.

In summary, the present invention demonstrates for the first time that the Cas13b can trans-cleave the non-natural sequence, and this property can be applied to nucleic acid detection. However, no one has previously applied for relevant patents using this feature.

In some embodiments, when the Cas13b protein is CcaCas13b, the non-natural sequence is ArUArUArU or rUArUArUA, and more preferably rUArUArUA.

In another aspect, the present invention provides use of a non-natural sequence in preparation of a reagent for detecting a target nucleic acid, where the non-natural sequence includes any one or more of the following: (1) a sequence containing both a deoxynucleotide and a ribonucleotide; (2) a sequence containing a deoxynucleotide and/or ribonucleotide, where the deoxynucleotide and/or ribonucleotide bears an artificially created modification that does not exist under natural conditions; and (3) a sequence containing a deoxynucleotide and/or ribonucleotide, where a backbone composed of the deoxynucleotide and/or ribonucleotide bears an artificially created modification that does not exist under natural conditions; and the reagent further includes a Cas13 protein and crRNA.

In some specific examples, the non-natural sequence contains both a deoxynucleotide and a ribonucleotide.

In some specific examples, the deoxynucleotides and ribonucleotides in the non-natural sequence are arranged alternately at intervals. That is, a deoxynucleotide is followed by a ribonucleotide, or a ribonucleotide is followed by a deoxynucleotide.

In yet another aspect, the present invention provides a probe that can be trans-cleaved by a Cas13 protein guided by crRNA.

In some embodiments, after the Cas13 protein binds to the target RNA, the probe of the non-natural sequence can be trans-cleaved by the Cas13 protein. In some embodiments, the probe is 2-100 bp in length.

Further, the probe includes a non-natural sequence, where the non-natural sequence includes any one or more of the following: (1) a sequence containing both a deoxynucleotide and a ribonucleotide; (2) a sequence containing a deoxynucleotide and/or ribonucleotide, where the deoxynucleotide and/or ribonucleotide bears an artificially created modification that does not exist under natural conditions; and (3) a sequence containing a deoxynucleotide and/or ribonucleotide, where a backbone composed of the deoxynucleotide and/or ribonucleotide bears an artificially created modification that does not exist under natural conditions.

Further, the non-natural sequence in the probe is composed of nucleotides constituting RNA and nucleotides constituting DNA. The nucleotides constituting the RNA are any one or more of uracil ribonucleotide (rU), adenine ribonucleotide (rA), cytosine ribonucleotide (rC) and guanine ribonucleotide (rG), and the nucleotides constituting the DNA are any one or more of thymine deoxynucleotide (T), adenine deoxynucleotide (A), cytosine deoxynucleotide (C) and guanine deoxynucleotide (G).

Further, the probe is arranged in the order of ribonucleotide-deoxynucleotide or deoxynucleotide-ribonucleotide. The ribonucleotide is any one or more of a uracil ribonucleotide, an adenine ribonucleotide, a cytosine ribonucleotide and a guanine ribonucleotide, and the deoxynucleotide is any one or more of a thymine deoxynucleotide, an adenine deoxynucleotide, a cytosine deoxynucleotide and a guanine deoxynucleotide.

In some embodiments, the nucleotides are arranged sequentially in the order of rRD or DrR, where the rR is any one or more of rU, rA, rC and rG, and the D is any one or more of T, A, C and G.

Further, the probe sequence contains the following non-natural sequences: any one or more of rUArUArUA, ArUArUArU, rUrUrArUrUrU, TrUTrUTrU, ArAArAArA, CrCCrCCrC and GrGGrGGrG, where the rU is a uracil ribonucleotide, the A is an adenine deoxynucleotide, the T is a thymine deoxynucleotide, the rA is an adenine ribonucleotide, the C is a cytosine deoxynucleotide, the rC is a cytosine ribonucleotide, the G is a guanine deoxynucleotide, and the rG is a guanine ribonucleotide.

It can be understood that according to the nucleotide arrangement rules of the non-natural sequence, it can be calculated that there are 4ⁿ (n=the number of nucleotides in the non-natural sequence) types of non-natural sequences, e.g. a single chimera (poly ArA), a double chimera (poly rUArUA), a multiple chimera (UrACrGTrA), etc. Any non-natural sequence can be used for preparation of a probe to detect a target nucleic acid and is within the claimed scope of the present invention. Since the types of non-natural sequences are endless, the present invention designs 2 representative non-natural sequences and uses these 2 sequences for subsequent research.

Further, the probe sequence includes any one or more of rUArUArUA (poly rUA) and ArUArUArU (poly ArU).

In the present invention, a LwCas13a protein and a CcaCas13b protein are selected to cleave the above 2 chimeric sequences respectively. It is found that only the CcaCas13b protein can cleave the chimeric sequences, while the LwCas13a protein cannot. This may be because during evolution, the LwCas13a only has the characteristics of RNase. Meanwhile, the cleavage efficiency of poly rUA by the CcaCas13b protein can almost reach the level of cleaving the poly rU sequence (ssRNA), indicating that poly rUA is expected to become a universal cleavage sequence for the Cas12b protein family.

Further, the non-natural sequence includes a labeling substance, and the labeling substance is a fluorescent labeling substance or other modifying substances such as a color-developing modifying substance. In some embodiments, the labeling substance is a fluorescent labeling substance, and the presence or quantity of the target nucleic acid is indicated by the intensity of fluorescence.

Further, the probe refers to a probe with a fluorescent group introduced at one end of a non-natural sequence and a quenching group connected to the other end. The working principle of it is as follows: under normal circumstances, the fluorescence emitted by the fluorescent group is quenched by the quenching group due to the small distance between the fluorescent group and the quenching group. After the Cas protein cleaves the probe, the fluorescent group is separated from the quenching group, and the fluorescent group emits fluorescence normally, which is detected by an instrument, thereby achieving the purpose of detecting the target nucleic acid.

In yet another aspect, the present invention provides a system for detecting a target nucleic acid, including the above probe including a non-natural sequence, a Cas protein and crRNA. The crRNA can bind to the target RNA, thereby activating the Cas protein. The activated Cas protein cleaves the target sequence and also non-specifically trans-cleaves the probe to achieve the effect of detecting the target nucleic acid. The target RNA can be the target nucleic acid itself. When the target nucleic acid is DNA, the target RNA is transcribed from the target nucleic acid, and the crRNA is designed according to the target RNA sequence and the detection purpose.

Further, the Cas protein belongs to a Cas13 protein family.

Further, the Cas protein belongs to a Cas13a or Cas13b subfamily.

Further, the Cas protein is CcaCas13b.

Further, when the Cas protein is CcaCas13b, the probe sequence including rUArUArUA has a better effect.

Further, the target nucleic acid is DNA or RNA. When the target nucleic acid is RNA and the RNA concentration is not lower than 1 pM, the system can directly detect the target RNA. When the target nucleic acid is DNA, the DNA needs to be transcribed into RNA before subsequent detection.

In some embodiments, the transcription reaction is performed using a T7 polymerase.

In yet another aspect, the present invention provides a method for detecting a target nucleic acid, where the method conducts detection by using the system for detecting a target nucleic acid as described above, and the type of the target nucleic acid is DNA or RNA.

In some embodiments, the steps of the method are as follows: extracting DNA or RNA from the sample; and if the sample is RNA and the concentration is higher than 1 pM, directly detecting the RNA sample, and if the sample is DNA, requiring a transcription reaction to generate RNA, and then performing a detection reaction. A corresponding detection reaction system is prepared according to a detection object (DNA or RNA): 10 μL-20 μL of the above reaction solution is taken, and quickly transferred into a 96- or 384-well plate, and the plate is placed in a real-time fluorescence quantitative PCR instrument. Depending on the differences in the selected Cas13 protein, the reaction temperature of a CcaCas13b protein is 37° C. The data is read once every 30 s-60 s (preferably 60 s), and the reaction time is 30 min-2 h (preferably 60 min). After the reaction is completed, a real-time fluorescence curve is generated or the final fluorescence value is obtained by measurement, and whether the target nucleic acid exists in the sample is determined according to the fluorescence curve or the fluorescence value. Three replicates and a blank control are set for each experiment. If the sample concentration is lower than 1 pM, the sample can be pre-amplified to improve the detection sensitivity of the system.

In yet another aspect, the present invention provides use of a non-natural sequence in preparation of a reagent for improving efficiency of detecting target RNA, where the non-natural sequence is rUArUArUA, where the rU is a uracil ribonucleotide, and the A is an adenine deoxynucleotide. In some embodiments, the combination of the Cas13b protein and the rUArUArUA sequence has the best detection effect, producing the highest signal-to-noise ratio.

In yet another aspect, the present invention provides use of a non-natural sequence in preparation of a reagent for improving trans-cleavage efficiency of a Cas13 protein, where the non-natural sequence is rUArUArUA, where the rU is a uracil ribonucleotide, the A is an adenine deoxynucleotide, and the T is a thymine deoxynucleotide. The cleavage of the chimeric sequence by Cas13 is comparable to that of the conventional ssRNA sequence.

In summary, the present invention finds that, unlike the Cas13a protein, the Cas13b protein has the activity of cleaving sequences other than ssRNA. Specifically, the sequences other than ssRNA refer to chimeric sequences. Subsequently, the present invention confirms that the activity of the above Cas13b protein is comparable to its RNase activity. Therefore, based on the newly discovered properties of the Cas13b protein, the Cas13b protein and the chimeric sequence are combined to develop a multifunctional nucleic acid detection platform. The detection platform supports both auxiliary amplification technology and direct detection of the target nucleic acid. The detection limit of the platform reaches the pM level, and after introducing the pre-amplification step, the sensitivity of the platform is improved to the aM level.

Although the present invention only proves that the Cas13b protein has atypical trans-cleavage activity, this discovery also implies that other members of the Cas13 protein family may also have this activity or the ability to cleave other types of sequences. Moreover, the chimeric sequence used in the present invention takes synthetic analogs such as xeno nucleic acid (XNA) as a model, and the Cas13b protein can efficiently cleave the chimeric sequence, which also implies the potential of the Cas13b protein to cleave other types of non-natural nucleic acids. In conclusion, this atypical nuclease activity of the Cas13b protein not only increases the application methods of the Cas13 family in nucleic acid detection and biotechnology but also improves the possibility of diversified applications of non-natural nucleic acids.

The beneficial effects of the present invention include:
1. it is found that the Cas13b protein can cleave the non-natural sequence, and new use of this protein for nucleic acid (DNA and RNA) detection is provided;
2. the preference of the Cas13b protein for trans-cleaving the sequence is explored, and based on this preference, a corresponding detection system is designed. It is found that the detection effect of the Cas13b-chimeric sequence (preferably poly rUA) system is comparable to that of a conventional Cas13b-ssRNA system, and in specific cases, it is even better than a traditional detection method. The research results broaden the applications of the Cas13 protein and the non-natural sequence;
3. pre-amplification (isothermal amplification or PCR) of the detection sample can improve the detection efficiency of the Cas13-chimeric probe system, reaching the single-molecule level;
4. it further deepens researchers' understanding of the Cas13 and the non-natural sequences, providing new directions for the optimization of the CRISPR/Cas13 detection system; and
5. there are no nucleases in nature that can degrade non-natural sequences, so the half-lives of the non-natural sequences are longer than those of the DNA and RNA, and thus probes made from the non-natural sequences can be preserved for a longer time; and the detection stability of non-natural sequence probes is higher, and the detection results are more reliable.

### BRIEF DESCRIPTION OF THE DRAWINGS

To describe the technical solutions in the examples of the present invention or in the prior art more clearly, the following briefly describes the accompanying drawings required for describing the examples or the prior art. It is obvious that the accompanying drawings in the following description show some examples of the present invention, and a person of ordinary skill in the art may still derive other drawings from these accompanying drawings without creative efforts.

FIG. 1a: a schematic diagram of a workflow of a Cas13 protein trans-cleavage system;

FIG. 1b: sample detection in a purification process of a LwCas13a protein, where P (Pellet): a precipitate after ultrasonic lysis and centrifugation; S (Supernatant): a supernatant after ultrasonic lysis and centrifugation; W1 (Wash 1) and W2 (Wash 2): lysis buffers after the 1st and 2nd washes of Strep-Tactin resin before SUMO protease digestion, respectively; B (Beads): a protease lysis solution after incubation of SUMO protease with the Strep-Tactin resin; C: the finally obtained LwCas13a protein after concentration and purification; L: a protein marker. The black arrow points to the target band;

FIG. 1c: sample detection in a purification process of a CcaCas13b protein, where P: a precipitate after ultrasonic lysis and centrifugation; S: a supernatant after ultrasonic lysis and centrifugation; F (Flow-through): a flow-through solution of a chromatography column after incubation with Strep-Tactin resin before washing with a lysis buffer, aiming to detect whether there are a large number of protein products not captured by the resin; W1, W2, and W3: lysis buffers after the 1st, 2nd, and 3rd washes of the Strep-Tactin resin before SUMO protease digestion, respectively; B: a protease lysis solution after incubation of SUMO protease with the Strep-Tactin resin; C: the finally obtained CcaCas13b protein after concentration and purification; L: a protein marker. The black arrow points to the target band;

FIG. 1d: effects of the activated LwCas13a protein on trans-cleaving different reporters. NTC: non-target control, which is the negative control. Error bars represent the standard deviation, n=3.

FIG. 1e: effects of the activated CcaCas13b protein on trans-cleaving different reporters. NC: non-template control, which is the negative control. Error bars represent the standard deviation, n=3.

FIG. 2a: a sensitivity test of the activated CcaCas13b protein on trans-cleaving a TTATT sequence (single-stranded DNA, ssDNA). The concentration range of a DNA activator including a T7 promoter region is 1 nM-100 fM. NTC: non-template control, which is the negative control. Error bars represent the standard deviation, n=3.

FIG. 2b: a sensitivity test of the activated CcaCas13b protein on trans-cleaving a poly rA sequence. The concentration range of a DNA activator including a 17 promoter region is 1 nM-100 fM. NTC: non-template control, which is the negative control. Error bars represent the standard deviation, n=3.

FIG. 2c: a sensitivity test of the activated CcaCas13b protein on trans-cleaving a poly rU sequence. The concentration range of a DNA activator including a 17 promoter region is 1 nM-100 fM. NTC: non-template control, which is the negative control. Error bars represent the standard deviation, n=3.

FIG. 2d: a sensitivity test of the activated CcaCas13b protein on trans-cleaving a poly rUA sequence. The concentration range of a DNA activator including a 17 promoter region is 1 nM-100 fM. NTC: non-template control, which is the negative control. Error bars represent the standard deviation, n=3.

FIG. 2e: a sensitivity test of the activated CcaCas13b protein on trans-cleaving a poly ArU sequence. The concentration range of a DNA activator including a 17 promoter region is 1 nM-100 fM. NTC: non-template control, which is the negative control. Error bars represent the standard deviation, n=3.

FIG. 2f: a sensitivity test of the activated CcaCas13b protein on trans-cleaving a TArUrAUC sequence. The concentration range of a DNA activator including a T7 promoter region is 1 nM-100 fM. NTC: non-template control, which is the negative control. Error bars represent the standard deviation, n=3.

FIG. 3a: a schematic diagram of a working principle of a Cas13b-chimeric sequence detection system combined with amplification technology. When a detection target is DNA, primers with a 17 promoter fragment are used to amplify the DNA, and then an RNA activator is obtained through a 17 transcription reaction. When a detection target is RNA, a reverse-transcription reaction is first performed, followed by amplification and transcription reactions to obtain an RNA activator. The RNA activator will bind to crRNA in a Cas13b protein, thereby activating the Cas13b protein. The activated Cas13b protein trans-cleaves a chimeric sequence with physical or chemical modifications (such as fluorescent groups and biotin). Instruments and equipment are used to detect physical and chemical changes in a reaction system, and detection results are visualized.

FIG. 3b: a schematic diagram of a working principle of a Cas13b-chimeric sequence system for directly detecting a target RNA.

FIG. 4a: an effect of a CcaCas13b-poly rA system combined with amplification technology on detecting a target HPV 18 plasmid. Poly rA is used as the reporter and is simultaneously modified with a FAM fluorescent group and a quenching group. Before detection, the RPA technology is used to amplify the HPV 18 plasmid, with an amplification time of 20 min and a detection time of 60 min. NTC: non-template control, which is the negative control. Error bars represent the standard deviation, n=3.

FIG. 4b: an effect of a CcaCas13b-poly rU system combined with amplification technology on detecting a target HPV 18 plasmid. Poly rU is used as the reporter and is simultaneously modified with a FAM fluorescent group and a quenching group. Before detection, the RPA technology is used to amplify the HPV 18 plasmid, with an amplification time of 20 min and a detection time of 60 min. NTC: non-template control, which is the negative control. Error bars represent the standard deviation, n=3.

FIG. 4c: an effect of a CcaCas13b-poly rUA system combined with amplification technology on detecting a target HPV 18 plasmid. Poly rUA is used as the reporter and is simultaneously modified with a FAM fluorescent group and a quenching group. Before detection, the RPA technology is used to amplify the HPV 18 plasmid, with an amplification time of 20 min and a detection time of 60 min. NTC: non-template control, which is the negative control. Error bars represent the standard deviation, n=3.

FIG. 4d: an effect of a CcaCas13b-poly ArU system combined with amplification technology on detecting a target HPV 18 plasmid. Poly ArU is used as the reporter and is simultaneously modified with a FAM fluorescent group and a quenching group. Before detection, the RPA technology is used to amplify the HPV 18 plasmid, with an amplification time of 20 min and a detection time of 60 min. NTC: non-template control, which is the negative control. Error bars represent the standard deviation, n=3.

### DETAILED DESCRIPTION OF THE INVENTION

The present invention is further described in detail hereafter in conjunction with the accompanying drawings and specific examples. The examples are only used to explain the present invention and are not intended to limit the scope of the present invention. Based on the examples of the present invention, all other examples obtained by those of ordinary skill in the art without creative efforts shall fall within the scope of protection of the present invention.

Unless otherwise specified, the test methods used in the following examples are conventional methods. The materials, reagents, etc. used are shown in Table 1. Unless otherwise specified, the reagents and materials are commercially available. The nucleic acid sequences involved in the present invention are shown in Table 2, and all of them are synthesized by Integrated DNA Technologies (IDT) Company.

TABLE 1
Some reagents and manufacturers involved in the present invention

| Reagent | Manufacturer |
|---|---|
| pC013 TwinStrep-SUMO-LwaCas13a | Addgene #90097 |
| plasmid | |
| pC013 TwinStrep-SUMO-CcaCas13b | Addgene #182687 |
| plasmid | |
| T7 polymerase | Lucigen |
| rNTP mix | New England Biolabs |
| Murine RNase inhibitor | New England Biolabs |
| Recombinase polymerase amplification kit | TwistDx |

TABLE 2
All sequences involved in the present invention

| | | Sequence |
|---|---|---|
| Sequence name | Specific sequence (5′-3′) | number |
| ssDNA reporter | /56-FAM/TTATT/3IABKFQ/ | SEQ ID NO: 1 |
| ssRNA polyrA | /56-FAM/rArArArArArA/3IABKFQ/ | SEQ ID NO: 2 |
| ssRNA poly rU | /56-FAM/rUrUrUrUrUrU/3IABKFQ/ | SEQ ID NO: 3 |
| LwCas 13a motif | /56-FAM/T*A*rArU*G*C/3IABKFQ/ | SEQ ID NO: 4 |
| reporter | | |
| CcaCas13b motif | /56-FAM/T*A*rUrAG*C*/3IABKFQ/ | SEQ ID NO: 5 |
| reporter | | |
| Chimeric poly rUA | /56-FAM/rUArUArUA/3IABKFQ/ | SEQ ID NO: 6 |
| Chimeric poly ArU | /56-FAM/ArUArUArU/3IABkFQ/ | SEQ ID NO: 7 |
| DNA activator sense | GAAATTAATACGACTCACTATAGGGTTGTTGGG | SEQ ID NO: 8 |
| | GTAACCAACTATTTGTTACTGTTGTTTATGTCA | |
| | TTATGTGCTGCCATATCTACTTCAGAAACTACA | |
| | TATAAAAATACT | |
| DNA activator anti- | AGTATTTTTATATGTAGTTTCTGAAGTAGATAT | SEQ ID NO: 9 |
| sense | GGCAGCACATAATGACATAAACAACAGTAACAA | |
| | ATAGTTGGTTACCCCAACAACCCTATAGTGAGT | |
| | CGTATTAATTTC | |
| LwCas13a | MKVTKVDGISHKKYIEEGKLVKSTSEENRTSER | SEQ ID NO: 10 |
| | LSELLSIRLDIYIKNPDNASEEENRIRRENLKK | |
| | FFSNKVLHLKDSVLYLKNRKEKNAVQDKNYSEE | |
| | DISEYDLKNKNSFSVLKKILLNEDVNSEELEIF | |
| | RKDVEAKLNKINSLKYSFEENKANYQKINENNV | |
| | EKVGGKSKRNIIYDYYRESAKRNDYINNVQEAF | |
| | DKLYKKEDIEKLFFLIENSKKHEKYKIREYYHK | |
| | IIGRKNDKENFAKIIYEEIQNVNNIKELIEKIP | |
| | DMSELKKSQVFYKYYLDKEELNDKNIKYAFCHF | |
| | VEIEMSQLLKNYVYKRLSNISNDKIKRIFEYQN | |
| | LKKLIENKLLNKLDTYVRNCGKYNYYLQVGEIA | |
| | TSDFIARNRQNEAFLRNIIGVSSVAYFSLRNIL | |
| | ETENENDITGRMRGKTVKNNKGEEKYVSGEVDK | |
| | IYNENKQNEVKENLKMFYSYDFNMDNKNEIEDF | |
| | FANIDEAISSIRHGIVHFNLELEGKDIFAFKNI | |
| | APSEISKKMFQNEINEKKLKLKIFKQLNSANVF | |
| | NYYEKDVIIKYLKNTKFNFVNKNIPFVPSFTKL | |
| | YNKIEDLRNTLKFFWSVPKDKEEKDAQIYLLKN | |
| | IYYGEFLNKFVKNSKVFFKITNEVIKINKQRNQ | |
| | KTGHYKYQKFENIEKTVPVEYLAIIQSREMINN | |
| | QDKEEKNTYIDFIQQIFLKGFIDYLNKNNLKYI | |
| | ESNNNNDNNDIFSKIKIKKDNKEKYDKILKNYE | |
| | KHNRNKEIPHEINEFVREIKLGKILKYTENLNM | |
| | FYLILKLLNHKELTNLKGSLEKYQSANKEETFS | |
| | DELELINLLNLDNNRVTEDFELEANEIGKFLDF | |
| | NENKIKDRKELKKFDTNKIYFDGENIIKHRAFY | |
| | NIKKYGMLNLLEKIADKAKYKISLKELKEYSNK | |
| | KNEIEKNYTMQQNLHRKYARPKKDEKFNDEDYK | |
| | EYEKAIGNIQKYTHLKNKVEFNELNLLQGLLLK | |
| | ILHRLVGYTSIWERDLRFRLKGEFPENHYIEEI | |
| | FNFDNSKNVKYKSGQIVEKYINFYKELYKDNVE | |
| | KRSIYSDKKVKKLKQEKKDLYIRNYIAHFNYIP | |
| | HAEISLLEVLENLRKLLSYDRKLKNAIMKSIVD | |
| | ILKEYGFVATFKIGADKKIEIQTLESEKIVHLK | |
| | NLKKKKLMTDRNSEELCELVKVMFEYKALE | |
| CcaCas13b | MKNIQRLGKGNEFSPFKKEDKFYFGGFLNLANN | SEQ ID NO: 11 |
| | NIEDFFKEIITRFGIVITDENKKPKETFGEKIL | |
| | NEIFKKDISIVDYEKWVNIFADYFPFTKYLSLY | |
| | LEEMQFKNRVICFRDVMKELLKTVEALRNFYTH | |
| | YDHEPIKIEDRVFYFLDKVLLDVSLTVKNKYLK | |
| | TDKTKEFLNQHIGEELKELCKQRKDYLVGKGKR | |
| | IDKESEIINGIYNNAFKDFICKREKQDDKENHN | |
| | SVEKILCNKEPQNKKQKSSATVWELCSKSSSKY | |
| | TEKSFPNRENDKHCLEVPISQKGIVFLLSFFLN | |
| | KGEIYALTSNIKGFKAKITKEEPVTYDKNSIRY | |
| | MATHRMFSFLAYKGLKRKIRTSEINYNEDGQAS | |
| | STYEKETLMLQMLDELNKVPDVVYQNLSEDVQK | |
| | TFIEDWNEYLKENNGDVGTMEEEQVIHPVIRKR | |
| | YEDKFNYFAIRFLDEFAQFPTLRFQVHLGNYLC | |
| | DKRTKQICDTTTEREVKKKITVFGRLSELENKK | |
| | AIFLNEREEIKGWEVFPNPSYDFPKENISVNYK | |
| | DFPIVGSILDREKQPVSNKIGIRVKIADELQRE | |
| | IDKAIKEKKLRNPKNRKANQDEKQKERLVNEIV | |
| | STNSNEQGEPVVFIGQPTAYLSMNDIHSVLYEF | |
| | LINKISGEALETKIVEKIETQIKQIIGKDATTK | |
| | ILKPYTNANSNSINREKLLRDLEQEQQILKTLL | |
| | EEQQQREKDKKDKKSKRKHELYPSEKGKVAVWL | |
| | ANDIKRFMPKAFKEQWRGYHHSLLQKYLAYYEQ | |
| | SKEELKNLLPKEVFKHFPFKLKGYFQQQYLNQF | |
| | YTDYLKRRLSYVNELLLNIQNFKNDKDALKATE | |
| | KECFKFFRKQNYIINPINIQIQSILVYPIFLKR | |
| | GFLDEKPTMIDREKFKENKDTELADWFMHYKNY | |
| | KEDNYQKFYAYPLEKVEEKEKFKRNKQINKQKK | |
| | NDVYTLMMVEYIIQKIFGDKFVEENPLVLKGIF | |
| | QSKAERQQNNTHAATTQERNLNGILNQPKDIKI | |
| | QGKITVKGVKLKDIGNFRKYEIDQRVNTFLDYE | |
| | PRKEWMAYLPNDWKEKEKQGQLPPNNVIDRQIS | |
| | KYETVRSKILLKDVQELEKIISDEIKEEHRHDL | |
| | KQGKYYNFKYYILNGLLRQLKNENVENYKVF | |
| LwCas13a-crRNA- | GAUUUAGACUACCCCAAAAACGAAGGGGACUAA | SEQ ID NO: 12 |
| DNA activator | AACUCUGAAGUAGAUAUGGCAGCACAUAAUG | |
| CcaCas13b crRNA- | TCTGAAGTAGATATGGCAGCACATAATGACGTT | SEQ ID NO: 13 |
| DNA activator | GGAACTGCTCTCATTTTGGAGGGTAATCACAAC | |
| T7-promoter-F | TAATACGACTCACTATAGGG | SEQ ID NO: 14 |
| T7-promoter-R | CCCTATAGTGAGTCGTATTA | SEQ ID NO: 15 |
| Synthetic DNA HPV | ATGGCTGATCCAGAAGGTACAGACGGGGAGGGC | SEQ ID NO: 16 |
| 18 plasmid | ACGGGTTGTAACGGCTGGTTTTATGTACAAGCT | |
| | ATTGTAGACAAAAAAACAGGAGATGTAATATCT | |
| | GATGACGAGGACGAAAATGCAACAGACACAGGG | |
| | TCGGATATGGTAGATTTTATTGATACACAAGGA | |
| | ACATTTTGTGAACAGGCAGAGCTAGAGACAGCA | |
| | CAGGCATTGTTCCATGCGCAGGAGGTCCACAAT | |
| | GATGCACAAGTGTTGCATGTTTTAAAACGAAAG | |
| | TTTGCAGGAGGCAGCAAAGAAAACAGTCCATTA | |
| | GGGGAGCGGCTGGAGGTGGATACAGAGTTAAGT | |
| | CCACGGTTACAAGAAATATCTTTAAATAGTGGG | |
| | CAGAAAAAG | |
| HPV18-E1-13-RPA- | GAAATTAATACGACTCACTATAGGGTCGGATAT | SEQ ID NO: 17 |
| Forward | GGTAGATTTTATTGATACACA | |
| HPV18-E1-13-RPA- | CATGCAACACTTGTGCATCATTGTGGACCT | SEQ ID NO: 18 |
| Reverse | | |
| HPV18-E1- | UGCUGUCUCUAGCUCUGCCUGUUCACAAAAGUU | SEQ ID NO: 19 |
| CcaCas 13b-crRNA | GGAACUGCUCUCAUUUUGGAGGGUAAUCACAAC | |

### Example 1: Exploration of Preferences of Cas13 Proteins for Trans-Cleaving Nucleic Acid Sequences

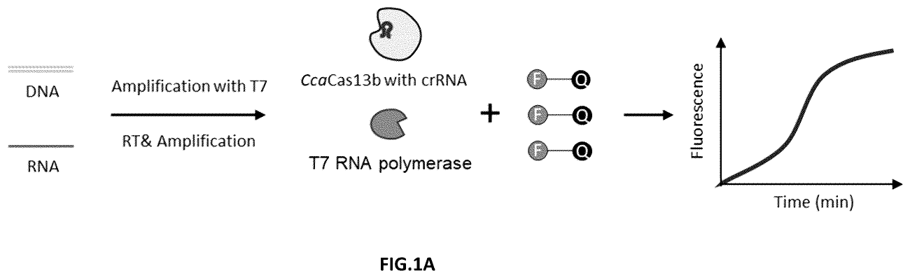

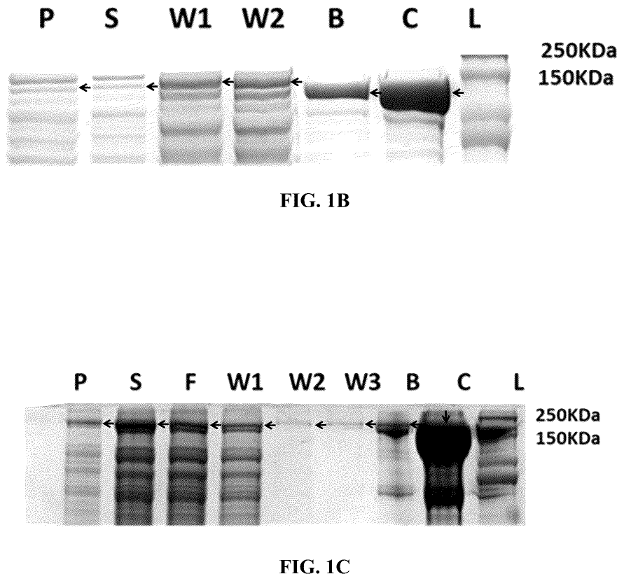

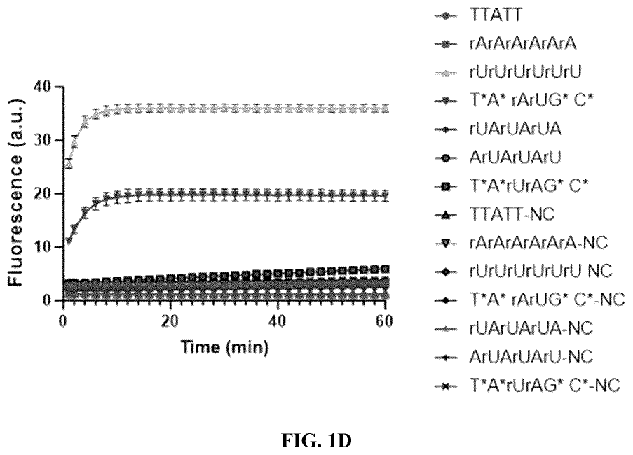

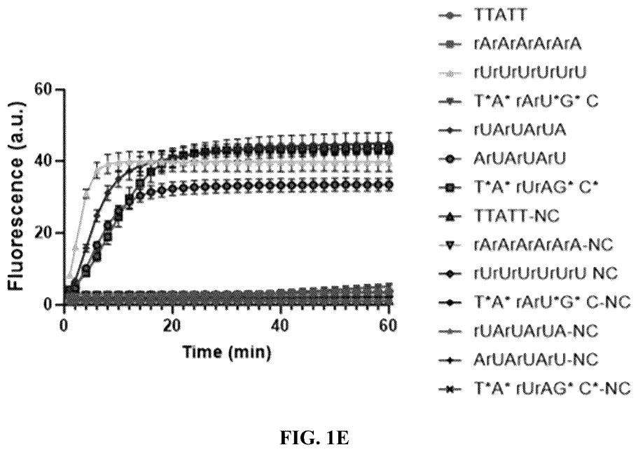

When the Cas13 protein family performs non-specific (trans) cleavage, it has different preferences for different RNA sequences. For example, LwCas13a and CcaCas13b hardly cleave the poly rA (rArArArArArA) sequence. Therefore, it is very important to explore the preferences of Cas13 protein variants for trans-cleavage before practical application. Thus, in this example, a cleavage detection system was constructed, which includes reporters (probes) (SEQ ID NOs: 1-7) modified with quenching groups and fluorescent genes, Cas13 proteins (LwCas13a and CcaCas13b proteins), and double-stranded DNA activators with a T7 promoter region (SEQ ID NOs: 8-9). The mechanism of action of the system is as follows: the DNA activator is first transcribed into an RNA activator, which can be recognized by Cas13 and activate the Cas13 protein. As long as the activated Cas13 protein has a preference for trans-cleaving the reporter in the system, it can degrade the reporter, separating the quenching group from the fluorescent group in the reporter. The fluorescent group, no longer inhibited by the quenching group, will emit fluorescence, which can then be detected by the instrument (FIG. 1a). It should be understood that when the content of DNA (activator) with the T7 transcription region is the same, the amount of the activated Cas13 protein is the same. The more sensitive the Cas13 protein is to the reporter (i.e., the higher the preference), the faster it cleaves the reporter. At the same time point before the plateau phase, the stronger the detected fluorescence, the higher the sensitivity of the detection system, and the shorter the detection time required. The specific operations for exploring the cleavage preferences of the Cas13 proteins in this example were as follows:

### 1.1 Design and Synthesis of Reporters (Probes)

In this example, a series of nucleic acid analogs (such as natural nucleic acid sequences and modified nucleic acid sequences) with a 56-FAM fluorescent group at the 5′ end and a 3IABkFQ quenching group at the 3′ end were designed and synthesized as reporters. The artificially synthesized reporter sequences for detecting the trans-cleavage activities of the Cas13 proteins included: ssDNA (TTATT, SEQ ID NO: 1), ssRNA (rArArArArArA (poly rA, SEQ ID NO: 2) and rUrUrUrUrUrU (poly rU, SEQ ID NO: 3)), the preferred cleavage sequence of LwCas13a (T*A*rArUG*C*, SEQ ID NO: 4), the preferred cleavage sequence of CcaCas13b (T*A*rUrAG*C*, SEQ ID NO: 5), and chimeric sequences (rUArUArUA (poly rUA, SEQ ID NO: 6) and ArUArUArU (poly ArU, SEQ ID NO: 7)), where T, A, and C represent thymidine deoxyribonucleotide, adenosine deoxyribonucleotide, and cytidine deoxyribonucleotide that make up DNA, respectively; rU and rA represent uridine ribonucleotide and adenosine ribonucleotide that make up RNA, respectively; and * represents phosphorothioate (PS) modification, which can interfere with the activity of nucleases.

### 1.2 Purification of Cas13 Proteins

In this example, the LwCas13a and CcaCas13b proteins were purified based on an existing method (14). Specifically:

### 1.2.1 Purification of the LwCas13a Protein

The pC013 TwinStrep-SUMO-LwaCas13a plasmid (the amino acid residue sequence of the LwaCas13a protein is shown in SEQ ID NO: 10) was transformed into Escherichia coli (E. coli) Rosetta 2 (DE3) competent cells. A single-colony of E. coli containing the above plasmid was inoculated into an LB liquid medium supplemented with 100 μg/mL ampicillin and cultured at 37° C. until the OD₆₀₀ of the bacterial solution reached 0.4-0.6. The bacterial solution was pre-cooled at 16° C. for 30 min, and then 0.1 mM IPTG was added to induce the expression of the exogenous protein for 18 h. After the induction was completed, the bacterial cells were collected and resuspended in 4× lysis buffer (20 mM Tris-HCl (pH=8.0), 0.5 M NaCl, 1 mM DTT, protease inhibitors), and then the bacteria were lysed on ice by ultrasonic treatment for 20 minutes. The lysed bacterial solution was centrifuged at 10,000 rpm for 60 min at 4° C., and the supernatant was collected. Strep-Tactin resin was added to the supernatant and incubated at 4° C. for 3 h. Subsequently, the Strep-Tactin resin bound with the target protein was loaded into a chromatography column and washed multiple times with cold lysis buffer (with the same components as described above). 20 μL of SUMO protease was added, and the reaction volume was supplemented to 3 mL with lysis buffer. The mixture was incubated at 4° C. for 18 h, and then the protease lysis solution was collected. Finally, the column was washed with 5 mL of lysis buffer, and the flow-through solution and the buffer were combined. The mixture was then dialyzed against 1×PBS buffer (pH=7.4) at 4° C. for 18 h. The dialyzed solution was concentrated using an Amicon Ultra-0.5 mL centrifugal filter column, and the protein was purified by Fast Performance Liquid Chromatography (FPLC). A small amount of the purified protein was taken and subjected to sodium dodecyl sulfate-polyacrylamide gel electrophoresis (SDS-PAGE) for detection. The results are shown in FIG. 1b. The remaining protein was stored in 1× storage solution (50 mM Tris-HCL pH 7.5, 600 mM NaCl, 5% Glycerol, 2 mM DTI) at −80° C. for more than 6 months.

### 1.2.2 Purification of the CcaCas13b Protein

The pC013 TwinStrep-SUMO-CcaCas13b plasmid (the amino acid residue sequence of the CcaCas13b protein is shown in SEQ ID NO: 11) was transformed into E. coli Rosetta 2 (DE3) competent cells. A single-colony of E. coli containing the above plasmid was inoculated into an LB liquid medium supplemented with 100 μg/mL ampicillin and cultured at 37° C. until the OD₆₀₀ of the bacterial solution was approximately 0.6. The bacterial solution was pre-cooled at 16° C. for 30 min, and then 0.1 mM IPTG was added to induce the expression of the exogenous protein overnight. After the induction was completed, the bacterial cells were collected and resuspended in 4× lysis buffer (20 mM Tris-HCl (pH=8.0), 0.5 M NaCl, 1 mM DTT, protease inhibitors), and then the bacteria were lysed on ice by ultrasonic treatment for 20 min. The lysed bacterial solution was centrifuged at 10,000 rpm for 60 min at 4° C., and the supernatant was collected. Strep-Tactin resin was added to the supernatant and incubated at 4° C. for 3 hours. Subsequently, the Strep-Tactin resin bound with the target protein was loaded into a chromatography column and washed multiple times with cold lysis buffer (with the same components as described above). 20 μL of SUMO protease was added, and the reaction system was supplemented to 3 mL with lysis buffer. The reaction was performed at 4° C. overnight, and then the protease lysis solution was collected. Finally, the column was washed with 5 mL of lysis buffer, and the protease lysis solution containing the target protein and the buffer were combined. The protein was then purified by Fast Performance Liquid Chromatography (FPLC). A small amount of the purified protein was subjected to sodium dodecyl sulfate-polyacrylamide gel electrophoresis (SDS-PAGE) for detection. The results are shown in FIG. 1c. The remaining protein was stored in 1× storage solution (50 mM Tris-HCL pH 7.5, 600 mM NaCl, 5% Glycerol, 2 mM DTI) at −80° C. for more than 6 months.

From the results of SDS-PAGE electrophoresis, a single and very dark band of the expected size (the LwCas13a protein was approximately 140 kDa in size, and the CcaCas13b protein was approximately 150 kDa in size) appeared in lane C of both FIG. 1b and FIG. 1c, indicating that the LwCas13a and CcaCas13b proteins were successfully purified, and the concentrated proteins had a high concentration, meeting the requirements for subsequent cleavage reactions.

### 1.3 Construction of a Cas13 Protein Trans-Cleavage System

The total reaction volume of the cleavage system was 20 μL. The components (with a volume of 18 μL) and their final concentrations of the system without the target to be detected or the activator were as follows: 1× reaction buffer (20 mM HEPES (pH=6.8) and 10 mM MgCl₂), 50 nM Cas13 protein (LwCas13a or CcaCas13b), 50 nM crRNA, 20 U murine RNase inhibitor, 1 mM rNTP mix, 0.125 U/μL T7 RNA polymerase, 250 nM fluorescent reporter (SEQ ID NO: 1 or SEQ ID NO: 2 or SEQ ID NO: 3 or SEQ ID NO: 4 or SEQ ID NO: 5 or SEQ ID NO: 6 or SEQ ID NO: 7). The sequence of crRNA was related to the type of Cas13 protein. Specifically, the crRNA sequence used for the LwCas13a protein is shown in SEQ ID NO: 12 of the sequence list, and that for the CcaCas13b protein is shown in SEQ ID NO: 13.

The sense strand (SEQ ID NO: 8) and the antisense strand (SEQ ID NO: 9) of the DNA activator were dissolved in PBS buffer with a final concentration of 5 nM. They were annealed at 95° C. for 5 min and then slowly cooled to room temperature at room temperature, and thus the DNA activator was prepared. In detail, in the experiment, the DNA activator with the 17 promoter region was recognized and transcribed by 17 RNA polymerase to generate an RNA sequence. Subsequently, the generated RNA sequence was recognized and bound by the crRNA in the Cas13b enzyme, thereby activating the cleavage function of the Cas13 enzyme. As a result, the activated Cas13 enzyme cis-cleaved the bound RNA sequence and trans-cleaved the free fluorescent reporters around it. 2 μL of the DNA activator was added to the above 18-μL reaction system. Then, the system was incubated at 37° C. for 60 min in a Roche LightCycler 480 II instrument. The fluorescence value of the reaction system was detected once every minute. Three replicates were set for each experimental group, and a negative control (i.e., nuclease-free water) was also set up simultaneously. The specific detection results are shown in FIG. 1d and FIG. 1e.

As could be seen from FIG. 1d, even when the concentration of the DNA activator was very high, the LwCas13a protein could not cleave sequences other than poly rU and its preferred motif (T*A*rArU*G*C). This indicates that the sequences that the LwCas13a protein can cleave are very limited, and the protein has relatively strong specificity. It was worth noting that although the cleavage speed of poly rU by the LwCas13a protein was similar to that of T*A*rArU*G*C, the fluorescence generated by the former was significantly higher than that of the latter, about 1.5 times higher. This may be because although the LwCas13a protein has a preference for sequences, compared with the T*A*rArU*G*C sequence, its cleavage activity on poly rU is better and the degradation is more complete.

As could be seen from FIG. 1e, different from the cleavage limitation of the LwCas13a protein, the CcaCas13b protein exhibited a different cleavage activity. In other words, it could not only cleave poly rU and its preferred motif (T*A*rUrA*G*C), but also cleave the chimeric sequences poly rUA and poly ArU. The sequences ranked from high to low in terms of the cleavage efficiency of the CcaCas13b protein were: poly rU>poly rUA>T*A*rUrA*G*C>poly ArU. Interestingly, whether in terms of cleavage efficiency or the intensity of the generated fluorescence, the effect of the CcaCas13b protein on cleaving poly rUA was better than that on cleaving poly ArU. This may be because the process of its recognition has a directional property during trans-cleavage. This result indicates, on the one hand, that chimeric sequences (poly rUA and poly ArU) composed of the same two nucleotides (rU and A) but with different nucleotide arrangements have different kinetics. On the other hand, it shows that the trans-cleavage activity of CcaCas13b is affected not only by the types of constituent units (nucleotides) of the sequence but also by the arrangement order of these units.

Considering the trans-cleavage abilities of the LwCas13a protein and the CcaCas13b protein, when it comes to cleaving chimeric sequences composed of two nucleotides (dual-chimeric sequences), LwCas13a has a better effect on cleaving poly ArU, while CcaCas13b is better at cleaving poly rUA. This indicates that different Cas13 proteins have different preferences for dual-chimeric sequences, that is, the types of chimeric sequences significantly affect the trans-cleavage of sequences by Cas13 proteins (FIG. 1d and FIG. 1e). Meanwhile, both LwCas13a and CcaCas13b have very low cleavage activities on the poly rA sequence but high activities on poly rU, which is consistent with previous results (9) and further proves that the types of nucleotides making up the sequence affect the trans-cleavage efficiency of Cas13 proteins. Moreover, these two Cas13 proteins cannot cleave ssDNA (TTATT), indicating that they do not have DNase activity.

In summary, the LwCas13a protein exhibits relatively specific RNase activity, while the CcaCas13b protein has atypical nuclease activity for trans-cleaving non-RNA sequences (i.e., chimeric sequences). In other words, different Cas13 proteins have different preferences for cleavage sequences. Specifically, when using the LwCas13a protein to detect target nucleic acids, poly rU is preferably used as the reporter (probe). When using the CcaCas13b protein to detect target nucleic acids, poly rU and poly rUA are preferably used as the reporters (probes). Since the fluorescence intensity generated by the CcaCas13b protein when cleaving poly rUA is higher than that when cleaving poly rU, it may indicate that CcaCas13b has stronger selectivity for poly rUA and degrades it more completely, so poly rUA is more preferred.

### Example 2: Sensitivity Test of the CcaCas13b Protein for Trans-Cleaving Different Nucleic Acid Sequences

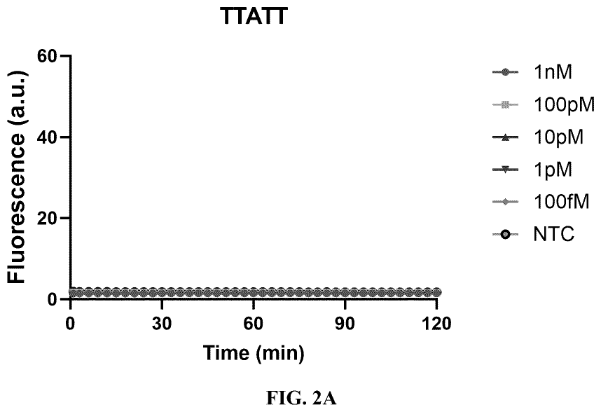

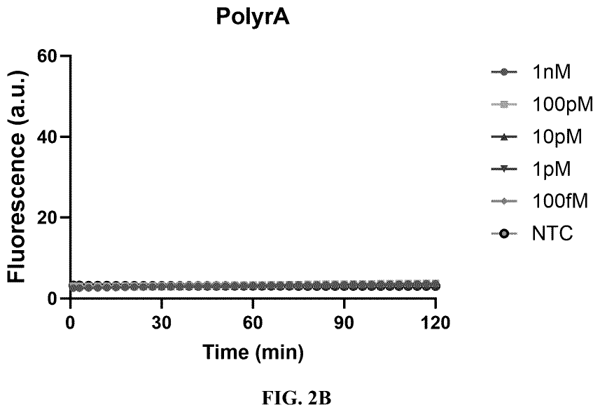

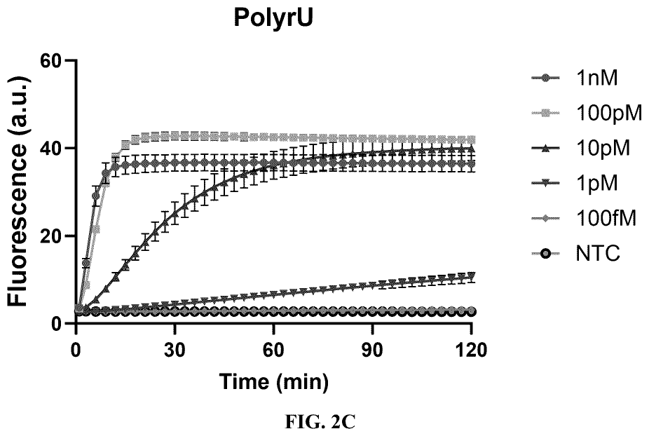

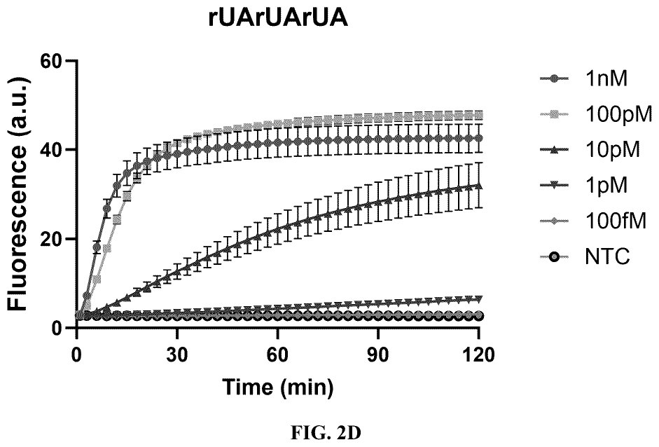

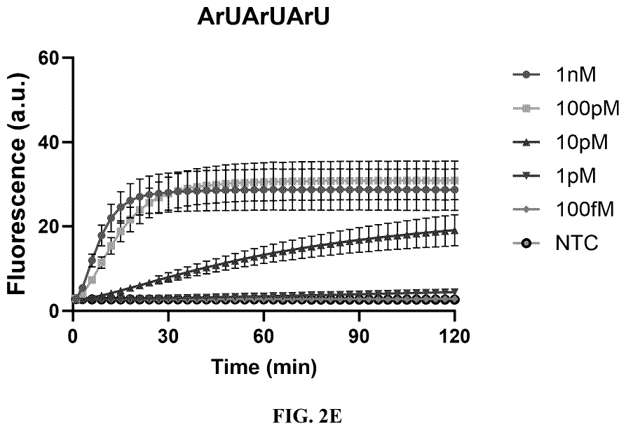

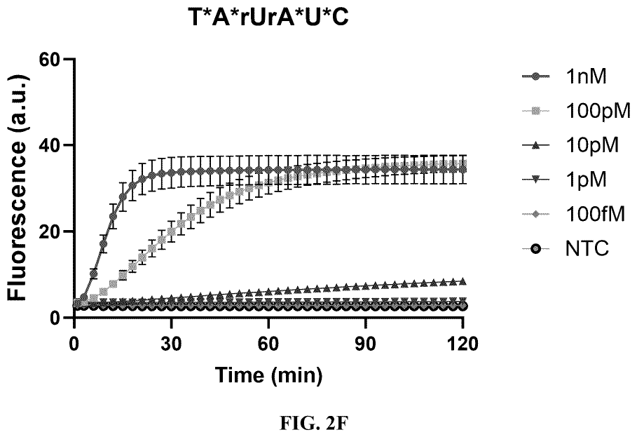

As shown in Example 1, compared with the relatively limited LwCas13a cleavage system, the CcaCas13b protein with atypical trans-cleavage enzyme activity can digest non-RNA sequences (including chimeric sequences). Therefore, to further explore the trans-cleavage function of the CcaCas13b protein, this example adopted DNA activators containing the T7 promoter region (SEQ ID NO: 1-SEQ ID NO: 3 and SEQ ID NO: 5-SEQ ID NO: 7) at different concentrations (1 nM, 100 pM, 10 pM, 1 pM, and 100 fM) to test whether the ability of CcaCas13b to cleave chimeric sequences (i.e., atypical trans-cleavage activity) is comparable to its ability to cleave typical RNA (i.e., RNase activity). In this example, the DNA activators were diluted to 1 nM, 100 pM, 10 pM, 1 pM, and 100 fM with PBS buffer respectively. Then, 2 μL of each was added to the reaction system. The Roche Light cycler 480 II instrument was used to detect fluorescence for 120 min. The remaining detection steps were the same as those described in Example 1. The detection results are shown in Table 3 and FIG. 2a-FIG. 2f.

TABLE 3
Detection sensitivity of the CcaCas13 system

| Nucleic acid | |
|---|---|
| sequence | Sensitivity |
| TTATT | N/A |
| poly rA | N/A |
| poly rU | 10 pM |
| poly rUA | 10 pM |
| poly ArU | 10 pM |
| T*A*rUrA*U*C | 100 pM |

From the results, it could be seen that regardless of the concentration of the DNA activator, the CcaCas13b protein could not cleave TTATT (ssDNA) and poly rA, which is consistent with the results in Example 1 (FIG. 1e). On the other hand, the detection limits of both the CcaCas13b protein+poly rU combination and the CcaCas13b protein+poly rUA combination were as high as 10 pM, followed by the CcaCas13b protein+poly ArU combination. It was worth noting that although the detection result of the poly ArU group in Example 1 was slightly worse than that of the T*A*rUrA*U*C motif sequence group (FIG. 1e), in the sensitivity test, the sensitivity of the former was 10 times that of the latter. This may be because although the motif sequence can be trans-cleaved, a relatively high concentration is required to achieve this, while the selection preference of poly ArU is stronger than that of the motif sequence, so it can be degraded even at a low concentration. This also reflects the potential of chimeric sequences in the field of nucleic acid detection. In addition, regardless of which reporter was used in the detection system, when the concentration of the DNA activator was 100 pM, the fluorescence value emitted by the detection system was the highest. This is because under this condition, the transcription reaction involving T7 polymerase can generate RNA that is 100 times the concentration of the DNA activator, thus reaching the optimal reaction concentration. In comparison, a higher concentration of the DNA activator inhibited the transcription process, and a lower concentration resulted in an insufficient reaction. Therefore, when the concentration of the nucleic acid to be detected is 100 pM, the detection results are the most reliable.

In summary, this example confirms that in nucleic acid detection, the sensitivity of the CcaCas13b protein and chimeric sequence (preferably poly rUA) group is equivalent to that of the CcaCas13b protein and poly rU combination, indicating that the atypical nuclease activity of CcaCas13b protein is equivalent to its typical RNase activity. Therefore, the atypical nuclease activity of CcaCas13b can be used to create a novel detection system. This system adopts chimeric sequences (preferably poly rUA) as reporters (probes), which further expands the application of the CcaCas13b protein and is not limited to cleaving single-stranded RNA sequences.

### Example 3: Working Principle of a Cas13b-Chimeric Sequence Detection System

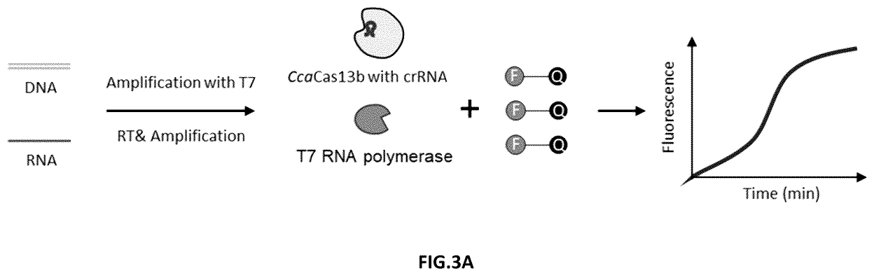

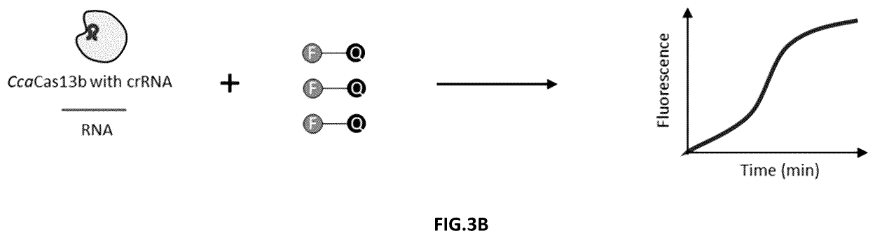

The traditional Cas13 nucleic acid detection is a system that recognizes RNA (activator) and cleaves RNA (reporter or probe). Examples 1 and 2 found that the CcaCas13b protein can trans-cleave chimeric sequences, that is, it has atypical trans-cleaving nuclease activity, and confirmed that the effect of the CcaCas13b protein in cleaving chimeric sequences is equivalent to that in cleaving traditional RNA. This means that researchers can construct a novel detection system based on the new characteristics of the Cas13b protein to complete the detection of target nucleic acids. In short, the detection system refers to replacing the original single-stranded RNA (ssRNA) probe with a chimeric sequence. This example would elaborate on the working principle of the detection system composed of the Cas13b protein and the chimeric sequence (Cas13b-chimeric sequence detection system) (FIG. 3a and FIG. 3b).

As could be seen from FIG. 3a, in the CRISPR detection system combined with amplification technology, primers containing T7 promoter sequences (SEQ ID NO: 14 and SEQ ID NO: 15) were first used to amplify the target nucleic acid (such as DNA and RNA) to produce amplicons. Amplification methods include isothermal amplification technology and Polymerase Chain Reaction (PCR). The isothermal amplification technology includes loop-mediated isothermal amplification (LAMP), recombinase polymerase amplification (RPA), strand displacement amplification (SDA), nucleic acid sequence-based amplification (NASBA), exponential amplification reaction (EXPAR), rolling circle amplification (RCA), etc. After the amplicons were generated, 17 polymerase transcribed the DNA amplicons with the 17 promoter region to generate RNA activators. The RNA activators were recognized by crRNA in the Cas13b protein, which in turn activated the Cas13b protein. The activated Cas13b protein trans-cleaved the modified chimeric sequences to obtain visualized detection results. The modifications were for visualizing the detection results. Therefore, it can be understood that as long as a modification can be visualized, it can be applied to the chimeric sequence. Commonly used modifications include physical or chemical modifications (such as fluorescent groups and biotin). It can be understood that the sensitivity of the CcaCas13b-chimeric sequence system provided by the present invention reaches the pM level, and adding an amplification procedure before detection enables the sensitivity of this detection system to be improved to the am level.

In addition, the Cas13b-chimeric sequence detection system could also directly detect the target RNA without an amplification procedure (FIG. 3b). In other words, when the target RNA had a certain concentration, it bound to the crRNA in the Cas13b protein, activating the Cas13b protein. Subsequently, the Cas13b protein cleaved the chimeric sequence. Instruments were used to detect and visualize the physical and chemical changes in the reaction system to obtain the final detection results.

### Example 4: Application of a Cas13b-Chimeric Sequence Detection System Combined with Amplification Technology

Example 3 introduced a novel CRISPR detection system based on the Cas13b protein and the chimeric sequence. However, it is still unclear what the effect of this detection system is in practical applications. Therefore, this example conducted a preliminary exploration on this issue. In the Cas13b-chimeric sequence detection system combined with amplification technology, this example first used the RPA technology to pre-amplify the artificially synthesized HPV 18 plasmid (SEQ ID NO: 16) for 20 minutes and then detected the target nucleic acid. The specific steps were as follows:

### 4.1 RPA Pre-Amplification

This example adopted the TwistAmp® Basic kit for RPA amplification. The operations were as follows: an amplification system was constructed, which included 29.5 μL of rehydration buffer, 2.4 μL of 10 μM amplification primer HPV18-E1-13-RPA-Forward (SEQ ID NO: 17), 2.4 μL of 10 μM amplification primer HPV18-E1-13-RPA-Reverse (SEQ ID NO: 18), 5 μL of HPV 18 plasmid (SEQ ID NO: 16), and 8.2 μL of nuclease-free water. The concentrations of the HPV18 plasmid were 1 aM, 100 aM, 1 fM, 10 fM, and 100 fM respectively. Then, 2.5 μL of 14 mM MgOAc was added to the above system to form a reaction unit with a total volume of 50 μL. The function of MgOAc was to initiate the amplification reaction. Finally, the constructed reaction unit was placed in a dry-heat bath and incubated at 37° C. for 20 min.

### 4.2 Detection of the Target Nucleic Acid

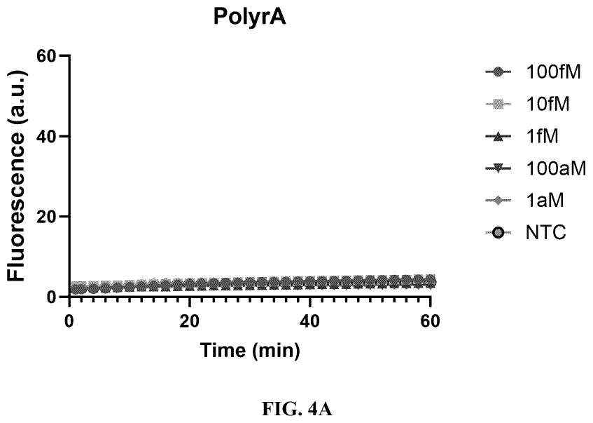

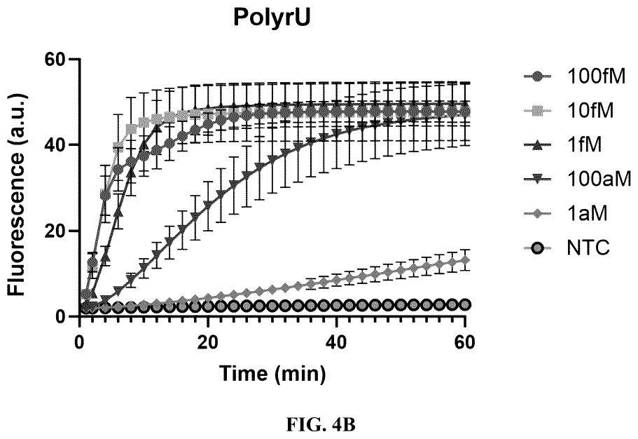

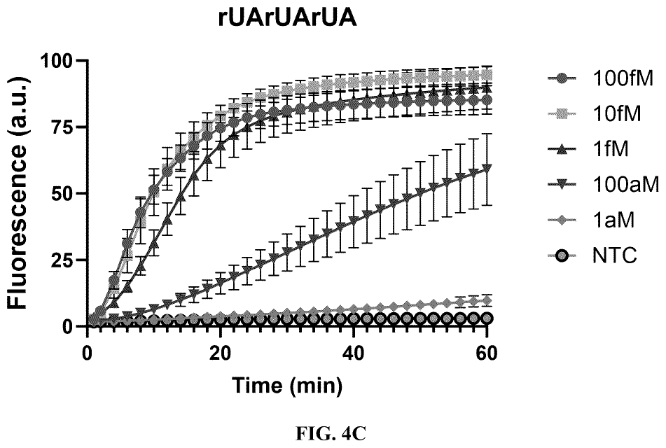

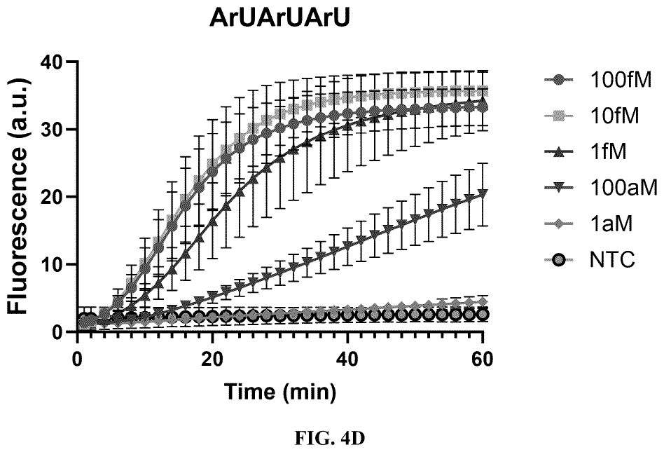

First, the detection system without the target to be detected or the activator as described in Example 1 was constructed. The reporters used in the detection of this example were ssRNA (poly rA and poly rU) and chimeric sequences (poly rUA and poly ArU). All reporters were modified with a FAM fluorescent group and a quenching group. The selected crRNA was HPV18-E1-CcaCas13b-crRNA (SEQ ID NO: 19). Then, 2 μL of the amplification product obtained in step “4.1” was pipetted and added to the detection system. The system was incubated at 37° C. for 60 min in a Roche Light cycler 480 II instrument, and the fluorescence was measured once every minute. The specific results are shown in Table 4 and FIGS. 4a-4d.

TABLE 4
Sensitivity of the CcaCas13 system
for detecting the HPV 18 plasmid

| Nucleic acid | |
|---|---|
| sequence | Sensitivity |
| poly rA | N/A |
| poly rU | 100 aM |
| poly rUA | 100 aM |
| poly ArU | 100 aM |

Due to the preference of the CcaCas13b protein, with the assistance of the RPA technology, when ssRNA was used as the reporter, the detection sensitivity of the CcaCas13b-poly rU system reached the aM level. In other words, the amplification technology could increase the sensitivity of the system by 10,000 times. In contrast, the CcaCas13b-poly rA system could not be used for the detection of target nucleic acids, and this result is consistent with the results of Examples 1 and 2. On the other hand, the sensitivity of the CcaCas13b-chimeric sequence (poly rUA or poly ArU) system also reached a level comparable to that of the CcaCas13b-poly rU system, and the detection time was within 60 minutes. In other words, it could achieve efficient and sensitive detection of target nucleic acids. The fluorescence emitted by the CcaCas13b-poly rUA system was significantly stronger than that of the CcaCas13b-poly rU system. This is beneficial for optimization, and a better non-natural nucleic acid sequence is selected to enhance the signal-to-noise ratio during low-concentration detection. Therefore, the poly-rUA sequence is preferably selected as the reporter.

In summary, this example confirmed that in practical detection applications, when the CcaCas13b-chimeric system was combined with amplification technology, its detection limit was comparable to that of the traditional system (CcaCas13b-poly rU system), both of which could reach the aM level. Even in specific cases, the detection effect of the former was even better than that of the latter.

### Example 5: A Detection System Based on Cas13 Proteins and Chimeric Sequences

Based on the exploration results of all the above examples, this example provided a detection system based on the Cas13 protein and chimeric sequence. In this example, the CcaCas13b protein was preferably used. The reaction volume of the system was 10 to 100 μL (20 μL was preferred in this example), and its components and final concentrations were as follows: 1× reaction buffer (20 mM HEPES (pH=6.8) and 10 mM MgCl₂), 50 nM Cas13 protein (the CcaCas13b protein was preferred in this example), 50 nM crRNA, 20 U murine RNase inhibitor, 1 mM rNTP mix, 0.125 U/μL T7 RNA polymerase, 250 nM reporter with a fluorescent group at the 5′ end and a quenching group at the 3′ end (poly rUA and poly ArU, and poly rUA was preferred in this example), and the nucleic acid sample to be detected.

### Example 6: A Method for Detecting Target Nucleic Acids Based on Cas13 Proteins and Chimeric Sequences

Combined with the results of the above examples, this example provided a detection method using the atypical trans-cleavage activity of Cas13 proteins, and the CcaCas13b protein was preferably used in this example. It should be understood that since the Cas13 protein can only be activated by RNA sequences, when the detection object is DNA, the DNA needs to be transcribed into RNA first, and then the subsequent detection steps can be performed. The specific steps of the method were as follows:

### 6.1 Pre-Amplification (Optional Step)

When the concentration of the sample to be detected was less than 1 pM, in order to improve the reliability of the detection results, the target nucleic acid needed to be amplified before the detection reaction. The amplification methods include but are not limited to PCR, LAMP, RPA, SDA, NASBA, EXPAR, and RCA, etc. In this example, the RPA technology was preferably used. The primers used for amplification needed to include the T7 promoter sequence (SEQ ID NO: 14 and SEQ ID NO: 15) to facilitate the subsequent transcription of the amplification products into RNA by 17 polymerase. It should be noted that when the detection object was DNA, it could be directly amplified using primers with the 17 sequence. During recognition, the T7 transcriptase transcribed the amplification products into RNA sequences, which were further amplified and recognized. When the detection object was RNA with a low concentration (i.e., less than 1 pM), the RNA needed to be reverse-transcribed into DNA, and then amplified, repeating the process mentioned before.

6.2 Construction of a detection System: when the concentration of the RNA sample to be detected was greater than 1 pM, the detection system could be directly constructed. The system was the same as that described in Example 6, and the volume of the sample to be detected was preferably 2 μL. The crRNA was designed according to the types of Cas13 proteins and the sequence of the target nucleic acid.

It should be understood that since the Cas13 protein can only be activated by RNA, if the object to be detected is DNA, a transcription reaction mediated by 17 polymerase is required to generate an RNA activator. The 17 polymerase can only transcribe after recognizing the 17 promoter sequence, and DNA itself does not carry the 17 promoter sequence. Therefore, the 17 promoter needs to be introduced into the detection system through PCR amplification technology or other means. Specifically, the primers used in PCR amplification carried the 17 promoter sequence. In other words, if the detection target was DNA, regardless of its concentration, an amplification step was required. In contrast, when the detection target was RNA, as long as the RNA concentration reached 1 pM, it could be directly detected.

6.3 Detection using a real-time fluorescence quantitative instrument: the detection time was preferably 60 minutes. The detection temperature was set according to the optimal enzymatic hydrolysis temperature of the Cas13 protein used in the reaction system, and 37° C. was preferred in this example.

6.4 Interpretation of results: after the detection reaction was completed, the real-time fluorescence quantitative instrument visualized the data and generated a time-fluorescence curve. If the trend of the curve was to rise first and then enter a plateau phase, the detection result was determined to be positive. If the curve was a relatively flat and horizontal straight line (with no obvious amplification trend), the detection result was negative. Alternatively, the determination can be made according to the final fluorescence value obtained from the detection. In other words, in the detection, a negative control and a positive control were set. The fluorescence value of the object to be detected was compared with those of the negative control and the positive control respectively. If its fluorescence value was close to that of the positive control, it was positive; otherwise, it was negative.

It should be understood that the core of the method described in this example lay in the combined use of the Cas13 protein and chimeric sequence. Based on this, probes and fluorescence detection methods were used in this example to detect target nucleic acids. If there are other supporting detection methods, they can also be used in combination with this combination.

The above has shown and described the basic principles, main features, and advantages of the present invention. For those skilled in the art, it is obvious that the present invention is not limited to the details of the above exemplary examples. Without departing from the spirit or essential features of the present invention, the present invention can be implemented in other specific forms. Therefore, the examples are to be regarded in any way as exemplary and non-limiting. The scope of the present invention is defined by the appended claims rather than the above description. Thus, all changes that fall within the meaning and scope of the equivalent elements of the claims are intended to be embraced within the present invention. Any reference signs in the claims should not be regarded as limiting the claims involved.

1. A probe comprising a non-natural sequence, wherein the non-natural sequence comprises any one or more of the following:
(1) a sequence containing both a deoxynucleotide and a ribonucleotide;
(2) a sequence containing a deoxynucleotide and/or ribonucleotide, wherein the deoxynucleotide and/or ribonucleotide bears an artificially created modification that does not exist under natural conditions; and
(3) a sequence containing a deoxynucleotide and/or ribonucleotide, wherein a backbone composed of the deoxynucleotide and/or ribonucleotide bears an artificially created modification that does not exist under natural conditions.

2. The probe according to claim 1, wherein the sequence of the probe comprises any one or more of rUArUArUA and ArUArUArU.

3. The probe according to claim 1, wherein the probe comprises a labeling substance, and the labeling substance is a fluorescent labeling substance or other modifying substances such as a color-developing modifying substance.

4. A kit for detecting a target nucleic acid in a sample, comprising: a Cas13 protein and a non-natural sequence capable of being trans-cleaved by the Cas13 protein.

5. The kit according to claim 4, wherein the non-natural sequence comprises any one or more of:
(1) a sequence containing both a deoxynucleotide and a ribonucleotide;
(2) a sequence containing a deoxynucleotide and/or ribonucleotide, wherein the deoxynucleotide and/or ribonucleotide bears an artificially created modification that does not exist under natural conditions; and
(3) a sequence containing a deoxynucleotide and/or ribonucleotide, wherein a backbone composed of the deoxynucleotide and/or ribonucleotide bears an artificially created modification that does not exist under natural conditions.

6. The kit according to claim 4, wherein the non-natural sequence comprises any one or more of rUArUArUA and ArUArUArU.

7. The kit according to claim 4, wherein the kit further comprises a necessary reagent required for a transcription reaction and/or amplification reaction and/or reverse transcription reaction.

8. The kit according to claim 4, wherein the kit further comprises crRNA.

9. A method for detecting a target nucleic acid, comprising: allowing a Cas13 protein to bind with RNA through an editable crRNA sequence; and trans-cleaving a non-natural sequence by the activated Cas13 with a non-RNase trans-cleavage ability, so as to indicate presence or quantity of the target nucleic acid by the number of cleaved non-natural sequences.

10. The method according to claim 9, wherein the target nucleic acid comprises DNA or RNA.

11. The method according to claim 9, wherein the Cas13 protein comprises a Cas13a protein or a Cas13b protein.

12. The method according to claim 9, wherein the Cas13 protein is a CcaCas13b protein.

13. The method according to claim 9, wherein the RNA is the target nucleic acid or is transcribed from the target nucleic acid.

14. The method according to any one of claim 9, wherein the non-natural sequence comprises any one or more of the following:
(1) a sequence containing both a deoxynucleotide and a ribonucleotide;
(2) a sequence containing a deoxynucleotide and/or ribonucleotide, wherein the deoxynucleotide and/or ribonucleotide bears an artificially created modification that does not exist under natural conditions; and
(3) a sequence containing a deoxynucleotide and/or ribonucleotide, wherein a backbone composed of the deoxynucleotide and/or ribonucleotide bears an artificially created modification that does not exist under natural conditions.

15. The method according to claim 9, wherein the non-natural sequence comprises a chimeric sequence.

16. The method according to claim 9, wherein the chimeric sequence comprises: a single chimera (poly ArA), a double chimera (poly rUArUA), and a multiple chimera (UrACrGTrA).

17. The method according to claim 9, wherein the non-natural sequence comprises any one or more of rUArUArUA and ArUArUArU.

18. The method according to claim 9, wherein when the target nucleic acid is natural DNA, the DNA needs to be transcribed to generate RNA.

19. The method according to claim 9, wherein the target nucleic acid is amplified before the RNA binds to the Cas13 protein.

20. The method according to claim 19, wherein when the target nucleic acid is RNA, the RNA needs to be reverse-transcribed into DNA and then amplified.
